(19) United States
(12) Patent Application Publication
(10) Pub. No.: US 2026/0224632 A1
(43) Pub. Date: Aug. 6, 2026

(54) USE OF A COMPOSITION COMPRISING A NEO-SYNTHESIZED EXTRACELLLAR MATRIX FOR THE TREATMENT OF CANCER, IN PARTICULAR FOR THE INHIBITION OF CANCER VIABILITY, MIGRATION, AND PROLIFERATION

(71) Applicant: NOVADIP BIOSCIENCES, Mont-Saint-Guibert (BE)

(72) Inventors: Denis DUFRANE, Mont-Saint-Guibert (BE); Hara EPISKOPOU, Mont-Saint-Guibert (BE)

(73) Assignee: NOVADIP BIOSCIENCES, Mont-Saint-Guibert (BE)

(21) Appl. No.: 19/147,802

(22) PCT Filed: Feb. 19, 2024
(86) PCT No.: PCT/EP2024/054107

§ 371 Date: Jul. 14, 2025

(30) Foreign Application Priority Data
Feb. 20, 2023 (BE) BE2023/5127

(51) Int. Cl. A61K 35/28; A61K 9/19; A61K 35/32; A61K 38/17; A61K 38/19; A61K 38/39; A61K 38/48; A61P 35/00

(52) U.S. Cl. CPC A61K 35/28; A61K 9/19; A61K 35/32; A61K 38/1709; A61K 38/191; A61K 38/39; A61K 38/482; A61P 35/00

(57) ABSTRACT

The present invention relates to the use of a composition comprising a neo-synthesized extracellular matrix for the treatment of cancer, in particular for the inhibition of cancer viability, migration and proliferation.

### FIELD OF THE INVENTION

The present invention relates to the use of the matrisome for the treatment of cancer, in particular for the inhibition of the viability, migration and proliferation of tumor cells. The present invention further relates to local implantation and local injections of matrisome for the treatment of cancer.

### BACKGROUND

WO2021105404A1 to Novadip Biosciences discloses sterile and desiccated biomaterials comprising devitalized differentiated cells having tissue regenerating and/or repairing properties. The biomaterials further comprise a particulate material. The cells and the particulate material are embedded in a neo-synthesized extracellular matrix. The particulate material is preferably gelatin, a ceramic material, or a demineralized bone matrix (DBM). Among a long list of disorders, also cancer is described, including a breast cancer, a skin cancer and a bone cancer. However, the inhibition of cancer is only exemplified in respect of the exosome. Consequently, only the anti-cancer use of the extracellular extract, i.e., the extracellular vesicles was exemplified.

WO2022112528A1 to Novadip Biosciences discloses cellular and/or extracellular extracts obtained from a scaffold-free 3-dimensional culture of mature cells and a particulate material for preventing and/or treating cancer. The mature cells secrete the neo-synthesized extracellular matrix. Both the mature cells and the particulate material are embedded in the neo-synthesized extracellular matrix. The extracts further comprise a pharmaceutically acceptable carrier. However, the inhibition of cancer is only exemplified in respect of the isolated extracellular vesicles, i.e. the supernatant, also referred to as the exosome. Consequently, only the anti-cancer use of the extracellular extract, i.e., the extracellular vesicles was exemplified.

### SHORT DESCRIPTION OF THE INVENTION

The present inventors have surprisingly found that the administration of desiccated and devitalized neo-synthesized extracellular matrix substantially inhibits the viability, migration and proliferation of cancer cells. The present inventors further found that the neo-synthesized extracellular matrix may act as a carrier for proteins and miRNAs that inhibit the viability, migration and proliferation of the cancer cells. Extracellular vesicles are usually only present as impurities, that means in very low amounts, such as less than 1 w % of the biomaterial prior to its desiccation and devitalization, preferably less than 0.1 w % of the biomaterial prior to its desiccation and devitalization, even more preferably less than 0.01 w % of the biomaterial prior to its desiccation and devitalization, even more preferably less than 0.001 w % biomaterial prior to its desiccation and devitalization.

Accordingly, a first aspect of the invention is a composition comprising:
devitalized differentiated cells;
3-dimensional neo-synthesized extracellular matrix as a vehicle of bioactive components selected from the group consisting of proteins, mRNAs, miRNAs, lipids, extracellular vesicles;
a particulate material;
   for use in the treatment or prevention of cancer;
   characterized in that:
the differentiated cells secreted the 3-dimensional neo-synthesized extracellular matrix prior to devitalization; and
the differentiated cells and the particulate material are embedded in the 3-dimensional neo-synthesized extracellular matrix.

In another aspect the composition comprises one or more of EMILIN1, DCN, HTRA1, TNFSF10, SFRP1.

In another aspect composition comprises the bioactive compounds in an amount of twice the amount, preferably five times the amount as compared to 2D culture of undifferentiated cells, preferably to 2D culture of undifferentiated ASCs.

In a preferred embodiment, the composition comprises:
devitalized differentiated cells;
3-dimensional neo-synthesized extracellular matrix as a vehicle of bioactive compounds selected from the group consisting of proteins, mRNAs, miRNAs, lipids, extracellular vesicles proteins, mRNAs, miRNAs, lipids, extracellular vesicles; and
a particulate material;
   for use in the treatment or prevention of cancer;
   characterized in that:
the differentiated cells secreted the 3-dimensional neo-synthesized extracellular matrix prior to devitalization;
the cells and the particulate material are embedded in the 3-dimensional neo-synthesized extracellular matrix;
the composition is substantially free of extracellular vesicles; and
preferably the particulate material is gelatin, and even more preferably gelatin beads.

In another aspect, the composition is used for:
inhibiting the viability of cancer cells,
inhibiting the proliferation of cancer cells;
inhibiting the migration of cancer cells;
inhibiting the cell colony formation of cancer cells;
or any combination thereof.

In another aspect, the composition is:
desiccated, preferably by lyophilization,
size reduced, preferably by grinding to volumetric particle size distribution range of 100 to 5000 micrometers as measured by laser diffraction granulometry; and
sterilized, preferably by gamma-irradiation.

In another aspect, the composition is free of external scaffolds.

In another aspect, the devitalized differentiated cells are stem cells-derived, in particular mesenchymal stem cells, preferably adipose tissue-derived stem cells.

In another aspect, the differentiated cells are selected from the group comprising or consisting of osteoblasts, osteocytes, chondroblasts, chondrocytes, keratinocytes, myofibroblasts, epithelial cells, endothelial cells, adipocytes, neural cells, and precursors thereof, and preferably are soft tissue cells, chondroblasts or osteoblasts.

In another aspect, the composition comprises the neo-synthesized extracellular matrix in a content of 0.001 w % to 10 w %, preferably from 0.01 w % to 7.5 w %, even more preferably from 0.1 w % to 5 w % as compared to the total weight of the composition.

In another aspect, the devitalized differentiated cells are derived from stem cells.

In another aspect, the devitalized differentiated cells are derived from stem cells selected from the group consisting of:
pluripotent stem cells (PSCs) such as embryonic stem cells (ESCs) or induced pluripotent stem cells (iPSCs);
adult stem cells such as hematopoietic stem cells (HSCs), skin stem cells (SSCs), neural stem cells (NSCs); and
mesenchymal stem cells (MSCs), preferably derivable from adipose tissue, peripheral blood or placenta,
   and preferably are mesenchymal stem cells.

In another aspect, the composition is scaffold-free.

In another aspect, the particulate material is selected from the group consisting of
an organic material, including demineralized bone matrix (DBM), gelatin, agar/agarose, alginates chitosan, chondroitin sulfate, collagen, elastin or elastin-like peptides (ELP), fibrinogen, fibrin, fibronectin, proteoglycans, heparan sulfate proteoglycans, hyaluronic acid, polysaccharides, laminins and cellulose derivatives;
calcium compound;
a polymer, including polyanhydrides, polylactic acid (PLA), poly(lactic-co-glycolic acid) (PLGA), polyethylene oxide/polyethylene glycol (PEO/PEG), poly(vinyl alcohol) (PVA), fumarate-based polymers such as, for example polypropylene fumarate) (PPF) or polypropylene fumarate-co-ethylene glycol) (P(PF-co-EG)), oligopolypthylene glycol) fumarate) (OPF), poly(aldehyde guluronate) (PAG), polyp-vinyl pyrrolidone) (PNVP), or combinations thereof;
a gel, including a self-assembling oligopeptide gel, a microgel, a nanogel, a particulate gel, a hydrogel, a thixotropic gel, a xerogel, a responsive gel, or combinations thereof;
a creamer; and
Any combinations thereof.

In another aspect, the particulate material is gelatin, and even more preferably gelatin beads.

In another aspect, the composition further comprises a pharmaceutically acceptable carrier.

In another aspect, the cancer is a solid cancer selected from the group consisting of s selected from the group comprising, or consisting of, a bone cancer, a brain cancer, a skin cancer, a breast cancer, a cancer of the central nervous system, a cancer of the cervix, a cancer of the upper aero digestive tract, a colorectal cancer, an endometrial cancer, a germ cell cancer, a bladder cancer, a kidney cancer, a laryngeal cancer, a liver cancer, a lung cancer, a neuroblastoma, an esophageal cancer, an ovarian cancer, a pancreatic cancer, a pleural cancer, a prostate cancer, a retinoblastoma, a small intestine cancer, a soft tissue sarcoma, a stomach cancer, a testicular cancer and a thyroid cancer, and preferably is bone cancer or any metastases thereof or skin cancer.

In another aspect, the composition is administered
as a local implant;
topically, preferably as a transdermal patch; or
by injection, preferably in the form of an injectable suspension.

Another aspect of the invention is a local implant, transdermal patch or injectable suspension comprising the composition of the invention for use in the treatment or prevention of cancer, in particular for use in the inhibition of the viability, migration and proliferation of cancer cells.

### DETAILED DESCRIPTION OF THE INVENTION

The invention is now described in further detail.

### Devitalized Differentiated Cells

The devitalized differentiated cells are embedded in the 3-dimensional neo-synthesized extracellular matrix.

In one embodiment, the differentiated cells are osteo-differentiated, skin-differentiated or chondro-differentiated cells. That means that differentiated cells have the ability to promote bone, skin and/or cartilage formation, and/or to maintain existing bone, skin and/or cartilage in a healthy physiological condition.

In one embodiment, the devitalized differentiated cells are derived from stem cells, such as pluripotent stem cells (PSCs), for example embryonic stem cells (ESCs) and induced pluripotent stem cells (iPSCs) or adult stem cells such as hematopoietic stem cells (HSCs), skin stem cells (SSCs), neural stem cells (NSCs), and mesenchymal stem cells (MSCs). MSCs are present in multiple tissues, including BM, adipose tissue, peripheral blood, and placenta, In a preferred embodiment, the devitalized differentiated cells are derived from mesenchymal stem cells, preferably adipose tissue-derived stem cells.

In one embodiment, the differentiated cells are selected from the group comprising or consisting of osteoblasts, osteocytes, chondroblasts, chondrocytes, keratinocytes, myofibroblasts, epithelial cells, endothelial cells, adipocytes, neural cells, and precursors thereof, and preferably are soft tissue cells, chondroblasts or osteoblasts.

In one embodiment, the osteogenic differentiation medium comprises or consists of DMEM supplemented with L-alanyl-L-glutamine (Ala-Gin, also called ‘Glutamax®’ or ‘Ultraglutamine®’), hPL, dexamethasone, ascorbic acid and sodium phosphate. In one embodiment, the osteogenic differentiation medium comprises or consists of DMEM supplemented with L-alanyl-L-glutamine, hPL, dexamethasone, ascorbic and sodium phosphate, and antibiotics, preferably penicillin, streptomycin, gentamycin and/or amphotericin B.

In certain embodiments, said cells are selected in a group comprising primary cells, stem cells, genetically modified cells, and a combination thereof.

After devitalization, usually at most 1% of said cells are viable, preferably at most 0.1%, even more preferably at most 0.01%, even more preferably at most 0.001% of the cells are viable.

The stem cells (examples: pluripotent stem cells (PSCs) such as embryonic stem cells (ESCs) and induced pluripotent stem cells (iPSCs) or adult stem cells such as hematopoietic stem cells (HSCs), skin stem cells (SSCs), neural stem cells (NSCs), and mesenchymal stem cells (MSCs)) preferably are mesenchymal stromal cells. Mesenchymal stromal cells may be obtained from bone marrow, adipose tissue, placenta, and blood. Mesenchymal stromal cells are capable of differentiating into different types of mesenchymal mature cells depending on the differentiation conditions.

In one embodiment, the differentiated cells are differentiated adipose tissue-derived stem cells (ASCs), preferably ASCs differentiated into cells selected from the group comprising or consisting of osteoblasts, chondrocytes, keratinocytes, myofibroblasts, epithelial, endothelial, connective, or neural cells and adipocytes.

In a some embodiments, ASCs are osteogenic differentiated ACSs, i.e. differentiated into osteogenic cells, in particular into osteoblasts.

In one embodiment, ASCs are differentiated into chondrogenic cells. In a particular embodiment, ASCs are differentiated into chondrocytes. In another embodiment, ASCs are keratinic differentiated ACSs. In other words, in one embodiment, ASCs are differentiated into keratinic cells. In a particular embodiment, ASCs are differentiated into keratinocytes.

In another embodiment, ASCs are myofibroblastic differentiated ACSs. In other words, in one embodiment, ASCs are differentiated into myofibroblastic cells. In a particular embodiment, ASCs are differentiated into myofibroblasts.

In another embodiment, ASCs are endothelial differentiated ACSs. In other words, in one embodiment, ASCs are differentiated into endothelial cells. In a particular embodiment, ASCs are differentiated into endothelial cells.

In another embodiment, ASCs are epithelial differentiated ACSs. In other words, in one embodiment, ASCs are differentiated into epithelial cells. In a particular embodiment, ASCs are differentiated into epithelial cells.

In another embodiment, ASCs are adipogenic differentiated ACSs. In other words, in one embodiment, ASCs are differentiated into adipogenic cells. In a particular embodiment, ASCs are differentiated into adipocytes. In another embodiment, ASCs are neural differentiated ACSs. In other words, in one embodiment, ASCs are differentiated into neural cells.

### Lyophilization

In one embodiment, the composition is desiccated, preferably by lyophilization.

### Size Reduction

After lyophilization, depending on the application, the particle size of the lyophilized composition can be reduced for example by grinding.

### Size Distribution

The particle size distribution after size-reduction can be determined by granulometry. Granulometry allows for the measurement of the size of the particle diameters.

### Laser Particle Size Measurement

A preferred measurement method is the laser particle size measurement. Laser particle size measurement allows the measurement of sizes between 0.05 and 900 μm.

The sample can be analyzed in solution (liquid route) or directly after lyophilization and optionally size reduction (dry route).

Wet laser particle size measurement allows the characterization of dispersions (elementary particle size after chemical dispersion) or suspended solids (“aggregate” particle size).

Dry laser particle size measurement allows the characterization of powders whose initial aggregation is not destroyed.

Preferably, the particle size is measured applying the wet method, using a Mastersizer equipment, which determines the particles size through laser diffraction. This technique is based on the measurement of angular intensity variations when the laser beam goes through the sample. Among the most important statistical parameters generated from a particle distribution analysis are the percentiles. These indicate in each case the size x below which a certain quantity of the sample (10% for Dx(10), 50% for Dx(50) and 90% for Dx(90)) by volume lies.

In one embodiment, the particle size is the Dx(50) by volume.

For example, a typical particle size may be measured applying wet leaser measurements using the Malvern Mastersizer. The Dx (10) (μm) indicates the size x below which 10% of total analyzed particles lies.

An exemplary particle size distribution according to the invention is:

Granulometry-liquid route: Dx (10) (μm) 365 Standard deviation: 38
Dx (50) (μm) 752 Standard deviation: 39
Dx (90) (μm) 1434 Standard deviation: 144

### Sterilization

In another embodiment, the composition is sterilized, preferably by gamma-irradiation.

In some embodiments, the freeze-drying of the biomaterial is performed at a temperature of about −80° C., preferably of about −50° C. under vacuum.

In practice, sterilization may be performed by any suitable method known from the state of the art, or a method adapted therefrom. Non-limitative examples of suitable methods include irradiation such as electron beam irradiation, X-ray irradiation, gamma-irradiation, or ultraviolet irradiation.

In certain embodiments, said sterile biomaterial is obtained by gamma-irradiation, preferably at a dose of about 7 kGy to about 45 kGy, more preferably at room temperature. Within the scope of the invention, the expression “about 7 kGy to about 45 KGy” encompasses 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 and 45 kGy.

In some embodiments, the biomaterial is obtained by gamma-irradiation at a dose of about 10 kGy to about 40 kGy. Within the scope of the invention, the term “room temperature” is intended to refer to a temperature comprised from about 15° C. to 25° C., preferably from 18° C. to about 22° C., which encompasses 18° C., 19° C., 20° C., 21° C. and 22° C. In some embodiments, room temperature is a temperature of about 20° C. The inventors observed that, in spite of the fact that sample undergoing gamma-irradiation have a general tendency to overheat and to potentially destroy valuable ingredients, gamma-irradiation of the biomaterial of the invention could be performed at room temperature without being substantially affected by overheating.

In some embodiments, the gamma-irradiation may be performed at a temperature below about 10° C., preferably on ice (about 0° C.). Within the scope of the invention, a temperature below about 10° C. encompasses 9.5° C., 8° C., 8.5° C., 8° C., 7.5° C., 7° C., 6.5° C., 6° C., 5° C., 4° C., 3° C., 2° C., 1° C., 0° C., −1° C., −2° C., −3° C., 4° C., −5° C., −10° C., −20° C., −30° C., −40° C., −50° C., −60° C., −70° C. and −80° C.

In practice, the gamma-irradiation may be performed for a duration that would depend from the size (e.g., expressed in mm3 or cm3) and/or the amount (e.g. expressed in mg or g) of biomaterial to be sterilized and/or the dose to be administered.

In certain embodiments, the gamma-irradiation may be performed from about 10 sec to about 24 h, preferably from about 5 min (300 sec) to about 12 h, more preferably, from about 10 min (600 sec) to about 3 h (10,800 sec). Within the scope of the invention, the expression “from about 10 sec to about 24 h” encompasses 10 sec, 12 sec, 14 sec, 16 sec, 18 sec 20 sec, 25 sec, 30 sec, 35 sec, 40 sec, 45 sec, 50 sec, 55 sec, 1 min, 1 min 30.2 min, 2 min 30.3 min, 3 min 30.4 min, 4 min 30.5 min, 6 min, 7 min, 8 min, 9 min, 10 min, 12 min, 14 min, 16 min, 18 min, 20 min, 22 min, 24 min, 26 min, 28 min, 30 min, 35 min, 40 min, 45 min, 50 min, 55 min, 1 h, 1 h 30, 2 h, 2 h 30, 3 h, 3 h 30, 4 h, 4 h 30, 5 h, 5 h 30, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h and 24 h.

### 3-Dimensional (3D) Neo-Synthesized Extracellular Matrix (ECM)

The 3-dimensional neo-synthesized extracellular matrix is an extracellular matrix that the differentiated cells surprisingly secrete when the particulate material is added.

The 3-dimensional neo-synthesized extracellular matrix serves as scaffold. Consequently, not external scaffold has to be added.

In one embodiment, due to the 3-dimensional neo-synthesized extracellular matrix, the differentiated cells—prior to devitalization—are different from 2-dimensional cell aggregates.

Thus, the differentiated cells are embedded in the 3-dimensional neo-synthesized extracellular matrix.

The cells embedded in the 3-dimensional neo-synthesized ECM secret certain proteins and miRNAs with anti-cancer activity.

After devitalization and optionally size reduction, the 3-dimensional neo-synthesized extracellular matrix is as a carrier for proteins and miRNAs with anti-cancer activity.

Accordingly, in certain embodiments, the 3-dimensional neo-synthesized extracellular matrix comprises one or more matrisomal proteins specific to soft tissue or calcified tissue.

In another embodiment, the 3-dimensional neo-synthesized extracellular matrix comprises one or more matrisomal proteins and miRNA molecules with anti-cancer activity.

Accordingly, the 3-dimensional neo-synthesized extracellular matrix can be used for the treatment or prevention of cancer, in particular for use in the inhibition of the viability, the migration or the proliferation of cancer cells.

Secreted Proteins with Anti-Cancer Activity

Examples of proteins with anti-cancer activity secreted by the differentiated cells present in the neo-synthesized extracellular matrix and implicated in the positive regulation of cell death pathways in osteosarcoma (OS) treatment such as apoptosis, autophagy and necroptosis include:

| | | Cell death pathways involved in OS |
|---|---|---|
| Protein name | Entry name | treatment (Gene Ontology): |
| Thrombospondin-1 | TSP1_HUMAN | positive regulation of execution |
| | | phase of apoptosis |
| Decorin | PGS2_HUMAN | positive regulation of execution |
| | | phase of apoptosis |
| Caveolin-1 | CAV1_HUMAN | positive regulation of cysteine-type |
| | | endopeptidase activity involved in |
| | | execution phase of apoptosis |
| Protein disulfide- | PDIA3_HUMAN | positive regulation of extrinsic |
| isomerase A3 | | apoptotic signaling pathway |
| Protein/nucleic acid | PARK7_HUMAN | positive regulation of extrinsic |
| deglycase DJ-1 | | apoptotic signaling pathway |
| 40S ribosomal protein | RS3_HUMAN | positive regulation of extrinsic |
| S3 | | apoptotic signaling pathway |
| Voltage-dependent | VDAC1_HUMAN | positive regulation of extrinsic |
| anion-selective | | apoptotic signaling pathway |
| channel protein 1 | | |
| Prolyl endopeptidase | SEPR_HUMAN | positive regulation of extrinsic |
| FAP | | apoptotic signaling pathway |
| High mobility group | HMGB1_HUMAN | positive regulation of extrinsic |
| protein B1 | | apoptotic signaling pathway |
| Protein PML | PML_HUMAN | positive regulation of extrinsic |
| | | apoptotic signaling pathway |
| ADP/ATP translocase 2 | ADT2_HUMAN | positive regulation of extrinsic |
| | | apoptotic signaling pathway |
| Astrocytic | PEA15_HUMAN | positive regulation of extrinsic |
| phosphoprotein PEA-15 | | apoptotic signaling pathway |
| Serine/threonine-protein | 2AAA_HUMAN | positive regulation of extrinsic |
| phosphatase 2A 65 kDa | | apoptotic signaling pathway |
| regulatory subunit A | | |
| alpha isoform | | |
| Serine/threonine-protein | PAK2_HUMAN | positive regulation of autophagy |
| kinase PAK 2 | | |
| Prostacyclin synthase | PTGIS_HUMAN | positive regulation of autophagy |
| Target of Myb protein 1 | TOM1_HUMAN | positive regulation of autophagy |
| Angiotensinogen | ANGT_HUMAN | positive regulation of autophagy |
| Catenin alpha-1 | CTNA1_HUMAN | positive regulation of autophagy |
| Protein LYRIC | LYRIC_HUMAN | positive regulation of autophagy |
| Mitogen-activated | MK03_HUMAN | positive regulation of autophagy |
| protein kinase 3 | | |
| Sorting nexin-18 | SNX18_HUMAN | positive regulation of autophagy |
| BAG family molecular | BAG3_HUMAN | positive regulation of autophagy |
| chaperone regulator 3 | | |
| Serine/threonine-protein | PP1A_HUMAN | positive regulation of autophagy |
| phosphatase PP1-alpha | | |
| catalytic subunit | | |
| Platelet-activating factor | PA1B2_HUMAN | positive regulation of autophagy |
| acetylhydrolase IB | | |
| subunit beta | | |
| Guanine nucleotide- | GNAI3_HUMAN | positive regulation of autophagy |
| binding protein G(k) | | |
| subunit alpha | | |
| Poly [ADP-ribose] | PARP1_HUMAN | positive regulation of autophagy |
| polymerase 1 | | |
| Endophilin-B1 | SHLB1_HUMAN | positive regulation of autophagy |
| E3 UFM1-protein | UFL1_HUMAN | positive regulation of autophagy |
| ligase 1 | | |
| Rho-associated protein | ROCK1_HUMAN | positive regulation of autophagy |
| kinase 1 | | |
| Heme oxygenase 1 | HMOX1_HUMAN | positive regulation of autophagy |
| La-related protein 1 | LARP1_HUMAN | positive regulation of autophagy |
| Optineurin | OPTN_HUMAN | positive regulation of autophagy |
| Sorting nexin-4 | SNX4_HUMAN | positive regulation of autophagy |
| Cyclin-Y | CCNY_HUMAN | positive regulation of autophagy |
| Transcription elongation | SPT5H_HUMAN | positive regulation of necroptotic |
| factor SPT5 | | process |

Accordingly, in another aspect, the composition of the present invention comprises one or more of the proteins mentioned in above table.

In another embodiment, the bioactive compounds are one or more of
Elastin microfibril interfacer 1 (EMILIN1),
Decorin (DCN),
Serine protease HTRA1,
Tumor necrosis factor TNFSF10, and/or
Secreted frizzled related protein 1 (SFRP1).

In a preferred embodiment, the composition comprises the bioactive compounds in an amount of twice the amount, preferably five times the amount as compared to 2D culture of undifferentiated cells, preferably as compared to 2D culture of undifferentiated ASCs.

In another embodiment, the composition comprises EMILIN1 in an amount of 100 times or more, preferably 200 times or more, even more preferably 400 times or more as compared to 2D culture of undifferentiated cells, preferably as compared to 2D culture of undifferentiated ASCs.

In another embodiment, the composition comprises DCN in an amount of 50 times or more, preferably 100 times or more, even more preferably 150 times or more as compared to 2D culture of undifferentiated cells, preferably as compared to 2D culture of undifferentiated ASCs.

In another embodiment, the composition comprises HTRA1 in an amount of 50 times or more, preferably 100 times or more, even more preferably 200 times or more as compared to 2D culture of undifferentiated cells, preferably as compared to 2D culture of undifferentiated ASCs.

In another embodiment, the composition comprises TNFSF10 in an amount of 2 times or more, preferably 5 times or more, even more preferably 7.5 times or more as compared to 2D culture of undifferentiated cells, preferably as compared to 2D culture of undifferentiated ASCs.

In another embodiment, the composition comprises SFRP1 in an amount of 10 times or more, preferably 25 times or more, even more preferably 50 times or more as compared to 2D culture of undifferentiated cells, preferably as compared to 2D culture of undifferentiated ASCs.

Secreted miRNAs with Anti-Cancer Activity

Examples of miRNAs with anti-cancer activity secreted by the differentiated cells and present in the neo-synthesized extracellular matrix are: hsa-miR-210-3p, hsa-miR-409-3p, hsa-let-7a-5p, hsa-miR-29b-3p, hsa-miR-30e-3p, hsa-let-7b-5p, hsa-miR-3184-3p, hsa-miR-92a-3p, hsa-miR-320a, hsa-miR-24-3p, hsa-let-7d-5p, hsa-miR-193b-5p, hsa-miR-361-3p, hsa-miR-199a-5p, hsa-miR-25-3p, hsa-miR-181a-5p, hsa-miR-151a-3p, hsa-miR-214-3p, hsa-miR-193a-5p, hsa-miR-30c-5p, hsa-miR-154-5p, hsa-let-7f-5p, hsa-miR-199a-3p, hsa-miR-664b-3p, hsa-miR-664a-5p, hsa-miR-3607-5p, hsa-miR-29a-3p, hsa-miR-27a-3p, hsa-miR-92b-3p, hsa-miR-199b-3p, hsa-miR-342-3p, hsa-miR-320b, hsa-miR-1291, hsa-let-7e-5p, hsa-miR-130a-3p, hsa-miR-3651, hsa-miR-103b, hsa-miR-1273g-3p, hsa-miR-30a-3p, hsa-miR-664b-5p, hsa-miR-34a-3p, hsa-miR-125a-5p, hsa-miR-145-5p, hsa-miR-664a-3p, hsa-miR-140-5p, hsa-miR-21-5p, hsa-miR-28-3p, hsa-miR-98-5p, hsa-miR-3609, hsa-let-7i-5p, hsa-miR-93-5p, hsa-miR-146b-5p, hsa-miR-374c-3p, hsa-miR-125b-5p, hsa-miR-34a-5p, hsa-miR-337-3p, hsa-miR-10a-5p, hsa-let-7g-5p, hsa-miR-222-3p, hsa-miR-4449, hsa-miR-22-3p, hsa-miR-191-5p, hsa-miR-3074-5p, hsa-miR-6516-3p, hsa-miR-4668-5p, hsa-miR-574-3p, hsa-miR-424-5p, hsa-let-7i-3p, hsa-miR-24-2-5p, hsa-miR-199b-5p, hsa-miR-424-3p, hsa-miR-103a-3p, hsa-miR-29b-1-5p, hsa-miR-423-5p, hsa-miR-328-3p, hsa-miR-324-5p, hsa-miR-335-5p, hsa-miR-574-5p, hsa-miR-17-5p, hsa-miR-660-5p, hsa-miR-425-5p, hsa-miR-23b-3p, hsa-miR-23a-3p, hsa-miR-185-5p, hsa-miR-4461, hsa-miR-196a-5p, hsa-let-7d-3p, hsa-miR-374b-5p, hsa-miR-127-3p, hsa-let-7c-5p, hsa-miR-423-3p, hsa-miR-196b-5p, hsa-miR-221-3p, hsa-miR-382-5p, hsa-miR-619-5p, hsa-miR-3613-5p, hsa-miR-3653-5p, hsa-miR-19b-3p, hsa-miR-99b-5p, hsa-miR-376c-3p, hsa-miR-99b-3p, hsa-miR-663b, hsa-miR-495-3p, hsa-miR-454-3p, and a combination thereof.

Accordingly, in another aspect, the composition of the present invention comprises one or more of above-mentioned miRNA(s) selected from a group consisting of.

Preferably, the composition of the present invention comprises one or more of miRNA(s) selected from a group consisting of: miRNA selected from the group consisting of MiR-140, miR-199a, miR-34a, miR-335 and miR-505.

Criteria and conventions for miRNA identification and nomenclature have been described in Ambros et al. (A uniform system for microRNA annotation. RNA 2003 9(3):277-279). The miRNAs sequences may be retrieved from the miRbase database (http://www.mirbase.org/) or the miRDB database (http://www.mirdb.org/).

In practice, the RNAs profile of the may be assessed by any suitable method known in the art, or any method adapted therefrom. Illustratively, RNA may be extracted, e.g. by the mean of commercial kit (such as miRNeasy kit from Qiagen®); and further sequenced, e.g. by the mean of a high-throughput sequencing system (such as NextSeq 500 system from Illumina®). Illustratively, one may use the Qiazol lysis reagent (Qiagen®, Hilden, Germany) and a Precellys homogenizer (Bertin® instruments, Montigny-le-Bretonneux, France). RNAs may be purified using Rneasy mini kit (Qiagen®, Hilden, Germany) with an additional on column DNase digestion according to the manufacturer's instruction.

Quality and quantity of RNA may be determined using a spectrophotometer (Spectramax® 190, Molecular Devices®, California, USA). cDNA may be synthesized from 0.5 μg of total RNA using RT RNA first strand kit (Qiagen®, Hilden, Germany) for genes expression profiles though customized PCR arrays (Customized Human Osteogenic and angiogenic RT² Profiler Assay—Qiagen®, Hilden, Germany). The ABI Quantstudio 5 system (Applied Biosystems®) and SYBR Green ROX Mastermix (Qiagen®, Hilden, Germany) may be used for detection of the amplification product. Quantification may be obtained according to the ΔΔCT method. The final result of each sample may be normalized to the means of expression level of housekeeping genes (e.g. ACTB, B2M and GAPDH).

### Scaffold-Free

Consequently, the composition of the present invention is scaffold-free. That means that no external, three-dimensional scaffold is used during differentiation.

### Particulate Material

The term “particulate material” as used herein refers to a solid material in the form of particles. Within the scope of the invention, “particulate material” includes organic materials, such as, e.g., demineralized bone matrix (DBM) and gelatin; ceramic materials; polymers, such as, e.g., polyanhydrides; gel, such as, e.g., hydrogel; and any combination thereof.

The particulate material is preferably selected from the group consisting of:
an organic material, including demineralized bone matrix (DBM), gelatin, agar/agarose, alginates chitosan, chondroitin sulfate, collagen, elastin or elastin-like peptides (ELP), fibrinogen, fibrin, fibronectin, proteoglycans, heparan sulfate proteoglycans, hyaluronic acid, polysaccharides, laminins and cellulose derivatives;
calcium compound;
a polymer, including polyanhydrides, polylactic acid (PLA), poly(lactic-co-glycolic acid) (PLGA), polyethylene oxide/polyethylene glycol (PEO/PEG), poly(vinyl alcohol) (PVA), fumarate-based polymers such as, for example polypropylene fumarate) (PPF) or polypropylene fumarate-co-ethylene glycol) (P(PF-co-EG)), oligopolypthylene glycol) fumarate) (OPF), poly(aldehyde guluronate) (PAG), polyp-vinyl pyrrolidone) (PNVP), or combinations thereof;
a gel, including a self-assembling oligopeptide gel, a microgel, a nanogel, a particulate gel, a hydrogel, a thixotropic gel, a xerogel, a responsive gel, or combinations thereof; or
a creamer; and
Any combinations thereof.

The particulate material preferably is gelatin, even more preferably gelatin beads.

In one embodiment, the gelatin of the invention is animal gelatin, preferably mammal gelatin, more preferably porcine gelatin.

As used herein, the term “porcine gelatin” may be replaced by “pork gelatin” or “pig gelatin”. A commercially available example is Cultispher. In one embodiment, the gelatin is porcine skin gelatin.

In certain embodiments, said gelatin is in the form of particles. The gelatin particles preferably particles have a volumetric mean diameter ranging from about 50 micrometers to about 1,000 micrometers as measured by laser diffraction granulometry, preferably with a Malvern Mastersizer. Within the scope of the invention, the expression “from about 50 micrometers to about 1,000 micrometers” encompasses 50 micrometers, 60 micrometers, 70 micrometers, 80 micrometers, 90 micrometers, 100 micrometers, 150 micrometers, 200 micrometers, 250 micrometers, 300 micrometers, 350 micrometers, 400 micrometers, 450 micrometers, 500 micrometers, 550 micrometers, 600 micrometers, 650 micrometers, 700 micrometers, 750 micrometers, 800 micrometers, 850 micrometers, 900 micrometers, 950 micrometers and 1,000 micrometers.

In one embodiment, gelatin is added at a concentration ranging from about 0.1 cm3 to about 5 cm3 for a 150 cm2 vessel, preferably from about 0.5 cm3 to about 4 cm3, more preferably from about 0.75 cm3 to about 3 cm3. In one embodiment, gelatin is added at a concentration ranging from about 1 cm3 to about 2 cm3 for a 150 cm2 vessel. In one embodiment, gelatin is added at a concentration of about 1 cm3, 1.5 cm3 or 2 cm3 for a 150 cm2 vessel. Within the scope of the invention, the expression “0.1 cm3 to about 5 cm3” encompasses 0.1 cm3, 0.2 cm3, 0.3 cm3, 0.4 cm3, 0.5 cm3, 0.6 cm3, 0.7 cm3, 0.8 cm3, 0.9 cm3, 1.0 cm3, 1.5 cm3, 2.0 cm3, 2.5 cm3, 3.0 cm3, 3.5 cm3, 4.0 cm3, 4.5 cm3 and 5.0 cm3.

In one embodiment, the particulate material is embedded in the secreted neo-synthesized extracellular matrix.

### Calcium-Based Particulate Material

In one embodiment, the particulate materials are ceramic particles. In one embodiment, the ceramic particles may be beads, powder, spheres, or microspheres.

In one embodiment, the ceramic particles of the invention are particles of calcium phosphate (CaP), calcium carbonate (CaCO3), calcium sulfate, or calcium hydroxide (Ca[OH]2), or combinations thereof.

Examples of calcium phosphate particles include, but are not limited to, hydroxyapatite (HA, Ca10(PO4)6(OH)2), tricalcium phosphate (TCP, Ca3[PO4]2), α-tricalcium phosphate (α-TCP, (α-Ca3(PO4)2), β-tricalcium phosphate (β-TCP, β-Ca3(PO4)2), tetracalcium phosphate (TTCP, Ca4(PO4)2O), octacalcium phosphate (Ca8H2(PO4)6.5H2O), amorphous calcium phosphate (Ca3(PO4) 2), hydroxyapatite/β-tricalcium phosphate (HA/β-TCP), hydroxyapatite/tetracalcium phosphate (HA/TTCP), and the like.

In one embodiment, the ceramic particles of the invention comprises or consists of hydroxyapatite (HA), tricalcium phosphate (TCP), hydroxyapatite/β-tricalcium phosphate (HA/β-TCP), calcium sulfate, or combinations thereof. In one embodiment, the ceramic material of the invention comprises or consists of hydroxyapatite (HA), β-tricalcium phosphate (β-TCP), hydroxyapatite/β-tricalcium phosphate (HA/β-TCP), α-tricalcium phosphate (α-TCP), calcium sulfate, or combinations thereof.

In one embodiment, the ceramic particles of the invention are particles of hydroxyapatite (HA). In another embodiment, the ceramic particles of the invention are particles of β-tricalcium phosphate (β-TCP). In another embodiment, the ceramic particles of the invention are particles of hydroxyapatite/β-tricalcium phosphate (HA/β-TCP). In other words, in one embodiment, the ceramic particles of the invention are a mixture of hydroxyapatite and β-tricalcium phosphate particles (called HA/β-TCP particles). In one embodiment, the ceramic particles of the invention consist of hydroxyapatite particles and β-tricalcium phosphate particles (called HA/β-TCP particles).

In one embodiment, the ceramic particles, preferably HA, β-TCP and/or HA/β-TCP particles, are not structured to form a predefined 3D shape or scaffold, such as for example a cube.

In one embodiment, the ceramic particles of the invention, preferably HA, —3-TCP and/or HA/β-TCP particles, are larger than about 50 μm, preferably larger than about 100 μm. In one embodiment, the ceramic particles of the invention, preferably HA, β-TCP and/or HA/β-TCP particles, have a mean diameter larger than about 50 μm, preferably larger than about 100 μm.

In one embodiment, the ceramic particles of the invention, preferably HA, β-TCP and/or HA/β-TCP particles, have a mean diameter of at least about 50 μm, preferably of at least about 100 μm, more preferably of at least about 150 μm. In another embodiment, the ceramic particles of the invention, preferably HA, TCP and/or HA/β-TCP particles, have a mean diameter of at least about 200 μm, preferably of at least about 250 μm, more preferably of at least about 300 μm.

In another embodiment, the ceramic particles of the invention, preferably HA, β-TCP and/or HA/β-TCP particles, have a mean diameter of at most about 2500 μm, preferably of at most about 2000 μm, more preferably of at most about 1500 μm. In one embodiment, the ceramic particles of the invention, preferably HA, β-TCP and/or HA/β-TCP particles, have a mean diameter of at most about 1000 μm, 900 μm, 800 μm, 700 μm or 600 μm.

In one embodiment, the ceramic particles of the invention, preferably HA, β-TCP and/or HA/β-TCP particles, have a mean diameter ranging from about 50 μm to about 1500 μm, preferably from about 50 μm to about 1250 μm, more preferably from about 100 μm to about 1000 μm. In one embodiment, the ceramic particles of the invention, preferably HA, β-TCP and/or HA/β-TCP particles, have a mean diameter ranging from about 100 μm to about 800 μm, preferably from about 150 μm to about 700 μm, more preferably from about 200 μm to about 600 μm.

In one embodiment, the HA/β-TCP particles have a volumetric mean diameter ranging from about 50 μm to about 1500 μm, preferably from about 50 μm to about 1250 μm, more preferably from about 100 μm to about 1000 μm. In one embodiment, the HA and β-TCP particles have a mean diameter ranging from about 100 μm to about 800 μm, preferably from about 150 μm to about 700 μm, more preferably from about 200 μm to about 600 μm as measured by laser diffraction granulometry, preferably with a Malvern Mastersizer.

In one embodiment, the ratio between HA and β-TCP (HA/β-TCP ratio) in the particles ranges from about 0/100 to about 100/0, preferably from about 10/90 to about 90/10, more preferably from about 20/80 to about 80/20. In one embodiment, the ratio HA/β-TCP in the particles ranges from about 30/70 to about 70/30, from about 35/65 to about 65/35, or from about 40/60 to about 60/40.

In one embodiment, the HA/β-TCP ratio in the particles is 0/100, i.e. the particles are particles of β-tricalcium phosphate. In another embodiment, the HA/β-TCP ratio in the particles is 100/0, i.e. the particles are particles of hydroxyapatite. In one embodiment, the HA/β-TCP ratio in the particles is about 10/90. In another embodiment, the HA/β-TCP ratio in the particles is about 90/10. In one embodiment, the HA/β-TCP ratio in the particles is about 20/80. In another embodiment, the HA/β-TCP ratio in the particles is about 80/20. In one embodiment, the HA/β-TCP ratio in the particles is about 30/70. In another embodiment, the HA/β-TCP ratio in the particles is about 70/30. In another embodiment, the HA/β-TCP ratio in the particles is about 35/65. In another embodiment, the HA/β-TCP ratio in the particles is about 65/35. In one embodiment, the HA/β-TCP ratio in the particles is about 40/60. In another embodiment, the HA/β-TCP ratio in the particles is about 60/40. In another embodiment, the HA/β-TCP ratio in the particles is about 50/50.

### Matrisome

“Matrisome” is used to describe the composition comprising, preferably consisting of:
devitalized differentiated cells;
3-dimensional neo-synthesized extracellular matrix as a carrier of bioactive compounds (proteins, mRNAs, miRNAs, lipids, extracellular vesicles);
a particulate material
   for use in the treatment or prevention of cancer
   characterized in that:
the differentiated cells secreted the neo-synthesized extracellular matrix prior to devitalization; and
the cells and the particulate material are embedded in the neo-synthesized extracellular matrix.

### Extracellular Vesicles

In one embodiment, the composition of the present invention or the matrisome are substantially free of extracellular vesicles. In one embodiment, the matrisome comprises less than 30 w %, preferably less than 20 w %, and even more preferably less than 10 w % of the extracellular vesicles as compared to the total dry weight of the composition.

In one embodiment, the composition comprises extracellular vesicles in a content of 0.001 w % to 10 w %, preferably from 0.01 w % to 7.5 w %, even more preferably from 0.1 w % to 5 w % as compared to the total dry weight of the composition.

In a preferred embodiment, extracellular vesicles are usually only present as impurities, that means in very low amounts, alternatively referred to as “substantially free” of extracellular vesicules. Preferably, extracellular vesicles are present in an amount of less than 1 w % of the biomaterial prior to its desiccation and devitalization, preferably less than 0.1 w % of the biomaterial prior to its desiccation and devitalization, even more preferably less than 0.01 w % of the biomaterial prior to its desiccation and devitalization, even more preferably less than 0.001 w % as compared to the total weight of the biomaterial prior to its desiccation and devitalization.

Accordingly, in a preferred embodiment, the extracellular vesicles are present in an amount from 0.01 w % to 1 w %, preferably from 0.001 w % to 1 w %, even more preferably from 0.0001 w % to 1 w % as compared to the total weight of the biomaterial prior to its desiccation and devitalization.

### Pharmaceutically Acceptable Carrier

In another aspect, the composition of the present invention comprises a pharmaceutically acceptable vehicle or carrier. As used herein, “pharmaceutically acceptable carrier” refers to any solvent, dispersion medium, coating, antibacterial and/or antifungal agent, isotonic and absorption delaying agent and the like.

The pharmaceutically acceptable carrier may comprise one or more ingredient(s) selected in a group of additives polypeptides; amino acids; lipids; and carbohydrates. Among carbohydrates, one may cite sugars, including monosaccharides, di-, tri-, tetra-, and oligosaccharides; derivatized sugars such as alditols, aldonic acids, esterified sugars and the like; and polysaccharides or sugar polymers.

Examples of suitable pharmaceutically acceptable vehicles may include polypeptides such as, e.g., gelatin, casein, and the like.

### Treatment or Prevention of Cancer

for use in the treatment or prevention of cancer, in particular for use in the inhibition of the viability, migration and proliferation of cancer cells

The terms “treatment”, “treating” or “alleviation” refer to therapeutic treatments wherein the object is to prevent or slow down (lessen) a cancer. A subject is successfully “treated” if, after receiving a therapeutic amount of a composition according to the present invention, the individual shows observable and/or measurable reduction in, or absence of cancer.

The treatment or use can be allogeneic, xenogeneic or autologous. Preferably, the treatment or use of present invention is allogenic.

“Allogeneic” or “allogenic” therapy means that the donor and the recipient are different individuals of the same species.

“Autologous” means that the donor and the recipient is the same individual.

“Xenogeneic” means that the donor is derived from an animal of a different species than the recipient.

The term “prevention” refers to preventing or avoiding the occurrence of symptom of a tissue disorder, including a skin disorder, bone disorder and/or cartilage disorder. In the present invention, the term “prevention” may refer to a secondary prevention, i.e. to the prevention of the re-occurrence of a symptom or a relapse of a tissue disorder, including a skin disorder, bone disorder and/or cartilage disorder. It may also refer, when the disease is cancer, such as, e.g., a bone cancer, to the occurrence of metastases after the treatment and/or the removal of a tumor. The term “effective amount” refers to an amount sufficient to effect beneficial or desired results including clinical results. An effective amount can be administered in one or more administrations.

### Cancer

The compositions of the present inventions are useful in the treatment or prevention of cancer. In one embodiment, the compositions of the present invention are useful in one or more of the following treatments:
inhibiting the viability of cancer cells,
inhibiting the proliferation of cancer cells;
inhibiting the migration of cancer cells;
inhibiting the cell colony formation of cancer cells; or
any combination thereof.

In one embodiment, the cancer is a solid cancer selected from the group consisting of s selected from the group comprising, or consisting of, a bone cancer, a brain cancer, a skin cancer, a breast cancer, a cancer of the central nervous system, a cancer of the cervix, a cancer of the upper aero digestive tract, a colorectal cancer, an endometrial cancer, a germ cell cancer, a bladder cancer, a kidney cancer, a laryngeal cancer, a liver cancer, a lung cancer, a neuroblastoma, an esophageal cancer, an ovarian cancer, a pancreatic cancer, a pleural cancer, a prostate cancer, a retinoblastoma, a small intestine cancer, a soft tissue sarcoma, a stomach cancer, a testicular cancer and a thyroid cancer, and preferably is bone cancer or any metastases thereof or skin cancer.

### Local Implant

In one embodiment, the composition of the present invention is administered as a local implant. Accordingly, another aspect of the invention is a local implant comprising the composition of the present invention.

### Patch

Another aspect of the invention is a patch, preferably a transdermal patch comprising the compositions of the present invention for use in the treatment or prevention of cancer, in particular for use in the inhibition of the viability of cancer cells.

Another aspect of the invention relates to a medical device comprising a composition according to the invention.

In certain embodiments, the medical device is a dressing for local application. In some embodiments, the dressing may comprise woven or non-woven fabrics.

In some embodiments, the medical device is coated by or with the composition according to the present invention. In certain embodiments, the medical device according to the invention is configured to allow the controlled release of the pharmaceutical composition. In some embodiments, the medical device is in the form of a patch.

### Liquid Injection

In another preferred embodiment, the composition is administered as an injection or in the form of an injectable liquid or an injectable suspension.

Accordingly, another aspect of the invention is an injectable liquid, wherein the composition of the invention is suspended.

Accordingly, in this embodiment, the size of the composition comprising the neo-synthesized extracellular matrix, is reduced for example through grinding or any other suitable size reduction method.

Usually, for injection purposes, the volumetric particle size of the composition is between 100 micrometers to 5000 micrometers, preferably, between 100 micrometers to 1000 micrometers, even more preferably from 100 micrometers to 500 micrometers as measured by laser diffraction granulometry, preferably with the Malvern Mastersizer.

In other embodiments, the volumetric particle size of the injectable composition is between 10 micrometers to 100 micrometers, preferably from 25 micrometers to 75 micrometers as measured by laser diffraction granulometry, preferably with the Malvern Mastersizer.

### Method of Treatment

Another aspect of the present invention is a method of treatment or prevention of cancer comprising the administration of a composition comprising:
devitalized differentiated cells;
3-dimensional neo-synthesized extracellular matrix as a vehicle of bioactive compounds, such as proteins, mRNAs, miRNAs, lipids, extracellular vesicles;
a particulate material, preferably gelatin
   for use in the treatment or prevention of cancer
   characterized in that:
the differentiated cells secreted the neo-synthesized extracellular matrix prior to devitalization; and
the cells and the particulate material are embedded in the neo-synthesized extracellular matrix.

### Manufacturing Process

The compositions of the invention may be manufactured in a scaffold-free process as shown in FIG. 1 comprising:
Stem cell collection;
Stem cell proliferation;
Osteogenic differentiation;
Particles sprinkling; and
3D-structure formation.

In one embodiment, the osteogenic differentiation medium comprises or consists of DMEM supplemented with L-alanyl-L-glutamine, PL, dexamethasone, ascorbic acid and sodium phosphate.

In one embodiment, the osteogenic differentiation medium comprises or consists of DMEM supplemented with L-alanyl-L-glutamine, hPL, dexamethasone, ascorbic and sodium phosphate, and antibiotics, preferably penicillin, streptomycin, gentamycin and/or amphotericin B.

In one embodiment, the osteogenic differentiation medium comprises or consists of DMEM supplemented with L-alanyl-L-glutamine, hPL (about 5%, v/v), dexamethasone (about 1 mM), ascorbic acid (about 0.25 mM) and sodium phosphate (about 2.93 mM). In one embodiment, the osteogenic differentiation medium comprises or consists of DMEM supplemented with L-alanyl-L-glutamine, hPL (about 5%, v/v), dexamethasone (about 1 pM), ascorbic acid (about 0.25 mM) and sodium phosphate (about 2.93 mM), penicillin (about 100 U/mL) and streptomycin (about 100 pg/mL). In one embodiment, the osteogenic differentiation medium further comprises amphotericin B (about 0.1%).

In one embodiment, the osteogenic differentiation medium consists of DMEM supplemented with L-alanyl-L-glutamine, hPL (about 5%, v/v), dexamethasone (about 1 pM), ascorbic acid (about 0.25 mM) and sodium phosphate (about 2.93 mM). In one embodiment, the osteogenic differentiation medium comprises or consists of DMEM supplemented with L-alanyl-L-glutamine, hPL (about 5%, v/v), dexamethasone (about 1 mM), ascorbic acid (about 0.25 mM) and sodium phosphate (about 2.93 mM), penicillin (about 100 U/mL), streptomycin (about 100 pg/mL) and amphotericin B (about 0.1%).

In another embodiment, the cells, in particular ASCs, are chondrogenic differentiated. In other words, in a preferred embodiment, the cells, in particular ASCs, are differentiated into chondrogenic cells. In still other words, in a preferred embodiment, the cells, in particular ASCs, are differentiated in chondrogenic medium. In a particular embodiment, the cells, in particular ASCs, are differentiated into chondrocytes.

In one embodiment, the obtained 3D structures are then desiccated, preferably by lyophilization (“matrisome”).

In one embodiment, the desiccated composition is then sterilized, preferably by gamma irradiation.

### SHORT DESCRIPTION OF THE DRAWINGS

FIG. 1 is a schematic representation of the manufacturing process of the composition of the present invention (matrisome).

FIG. 2 shows the effect of the compositions of the present invention (matrisome, NVDM2, Example 2) on the viability of three OS cell lines: 143B (FIG. 2A), SAOS2 (FIG. 2B), U2OS (FIG. 2C) in comparison to human bone marrow MSCs (BM-MSCs) (FIG. 2D). Doxorubicin (0.1 uM) was used as a positive control treatment and gelatin beads alone were used for comparison with NVDM2 treatment. All treatments were carried out in 96-well plates in transwells. The compositions of the invention (NVDM2) were administered in two different doses: 7 mg and 15 mg per well. The viability was measured using RealTime-Glo™ MT Cell Viability Assay at four different time points at 0, 24, 48, 72 hours after administration. The compositions of the present invention (matrisome, NVDM2) showed a dose-dependent inhibitory impact on the viability of the tested tumor cells which was significantly stronger than the gelatin beads alone. In contrast, the inhibitory effect observed on the viability BM-MSCs upon the treatment of matrisome was significantly lower than in case of OS cells and similar to the impact observed by the gelatin beads alone. Data represent the mean±s.d. values of % cell viability upon treatment normalized to the respective untreated sample derived from n number of different batches tested for each type of treatment.

FIG. 3 shows the effect of the compositions of the present invention (matrisome, NVDM2, Example 2) on the viability of two osteosarcoma cell lines: (A) 143B and (B) U2OS using CCK-8 viability assay. Doxorubicin (0.1 uM) was used as a positive control treatment and gelatin beads alone were used for comparison with NVDM2 treatment. All treatments were carried out in 96-well plates in transwells. The compositions of the invention (NVDM2) were administered in two different doses: 7 mg and 15 mg per well. The viability was measured using CCK-8 viability bioassay at 72 hours after administration. The compositions of the present invention (matrisome, NVDM2) showed a strong dose-dependent inhibitory impact on the viability of the tumor cells confirming the results presented in FIG. 2A. No impact on the viability of 143B cells was observed in the presence of gelatin beads alone (15 mg). Data represent the mean±s.d. values of % cell viability upon treatment normalized to the respective untreated sample. Blue line represents 100% of viability. Statistical significances between treated vs untreated samples at the respective time point were by ANOVA-I when values passed the normality tests (a=0.05) or by Kruskal-Walli's test when values did not pass the normality tests (a=0.05). *; p<0.01, *; p<0.001; ns, not significant. Blue dots: values of individual batches (biological replicates). Each chart represents the average value of the data from all batches tested. DS: lyophilized non irradiated powder of NVDM2; DP: lyophilized powder of NVDM2 after irradiation.

FIG. 4 shows the effect of the compositions of the present invention (matrisome, NVDM2, Example 2) on the viability of different types of tumor cell lines other than osteosarcoma (A) melanoma A375 cells, (B) breast carcinoma HS-578T cells, (C) lung carcinoma A549 cells and (D) U87 glioblastoma cells using CCK-8 viability assay. Doxorubicin (0.1 uM) was used as a positive control treatment and gelatin beads alone were used for comparison with NVDM2 treatment. All treatments were carried out in 96-well plates in transwells. The compositions of the invention (NVDM2) were administered in two different doses: 7 mg and 15 mg per well. The viability was measured using CCK-8 viability bioassay at 72 hours after administration. The compositions of the present invention (matrisome, NVDM2) showed a strong dose-dependent inhibitory impact on the viability of all the tested tumor cell lines, whereas no significant or much lower impact on the viability of the respective cells was observed in the presence of gelatin beads alone (15 mg). Data represent the mean±s.d. values of % cell viability upon treatment normalized to the respective untreated sample. Blue line represents 100% of viability. Statistical significances between treated vs untreated samples at the respective time point were tested by ANOVA-I. ***; p≤0.001; ns, not significant. Blue dots: values of individual batches (biological replicates). Each chart represents the average value of the data from all batches tested. DP: lyophilized powder of NVDM2 after irradiation.

FIG. 5 shows the effect of the compositions of the present invention (matrisome, NVDM2, Example 2) on the viability of non-tumor cell lines (A) human bone marrow mesenchymal stem cells (hBM-MSCs) and (B) adult human dermal fibroblasts (HDFa) using CCK-8 viability assay. Doxorubicin (0.1 uM) was used as a positive control treatment and gelatin beads alone were used for comparison with NVDM2 treatment. All treatments were carried out in 96-well plates in transwells. The compositions of the invention (NVDM2) were administered in two different doses: 7 mg and 15 mg per well. The viability was measured using CCK-8 viability bioassay at 72 hours after administration. The compositions of the present invention (matrisome, NVDM2) showed no impact on the viability of all the tested non-tumor cell line confirming the results presented in FIG. 2A, whereas a significant inhibitory impact was observed on the viability of the respective cells in the presence of doxorubicin. Data represent the mean±s.d. values of % cell viability upon treatment normalized to the respective untreated sample. Blue line represents 100% of viability. Statistical significances between treated vs untreated samples at the respective time point were tested by ANOVA-I. *, p≤0.05; **; p≤0.01, ***; p≤0.001; ns, not significant. Blue dots: values of individual batches (biological replicates). Each chart represents the average value of the data from all batches tested. DS: lyophilized non irradiated powder of NVDM2; DP: lyophilized powder of NVDM2 after irradiation.

FIG. 6 shows the effect of the compositions of the present (matrisome, NVDM2, Example 4) on the colony formation ability of two osteosarcoma (OS) cell lines: (A) 143B and (B) U2OS using CFU bioassay. Doxorubicin (0.005 uM and 0.01 uM) was used as a positive control treatment and gelatin beads alone were used for comparison with NVDM2 treatment. All treatments were carried out in 24-well plates in transwells. The compositions of the invention (NVDM2) were administered in two different doses: 10 mg and 50 mg per well. The compositions of the present invention (matrisome, NVDM2) showed a significant dose-dependent inhibitory impact on the colony forming ability of the OS cells, which was much stronger than in the presence of the gelatin beads alone (50 mg). Data represent the mean±s.d. values of % CFU (colony forming units) upon treatment normalized to the respective untreated samples. Blue line represents 100% of % CFU (colony forming units). Statistical significances between treated vs untreated samples at the respective time point were tested by ANOVA-I. *: p≤0.05; ***: p≤0.001, ns: not significant. Blue dots: values of individual batches (biological replicates). Each chart represents the average value of the data from all batches tested. DS: lyophilized non irradiated powder of NVDM2; DP: lyophilized powder of NVDM2 after irradiation.

FIGS. 7C and 7D show representative pictures of the colonies formed by 143B cells after 6 days of incubation with or without treatment (matrisome, NVDM2, Example 4). FIG. 7A shows the untreated 143B cells. FIG. 7B shows the comparative treatment with Doxorubicin at 0.005 uM. FIG. 7C shows the effect of the matrisome (NVDM2) at 10 mg. FIG. 7D shows the effect of the matrisome (NVDM2, Example 3) at 50 mg. FIG. 7E shows the comparative treatment with gelatin beads at 50 mg.

FIG. 8 shows the effect of the compositions of the present (matrisome, NVDM2, Example 4) on the migration ability of two osteosarcoma (OS) cell lines: (A) 143B and (B) U2OS using scratch/wound healing bioassay. Doxorubicin (1 uM) was used as a positive control treatment and gelatin beads alone were used for comparison with NVDM2 treatment. All treatments were carried out in 96-well plates in transwells. The compositions of the invention (NVDM2) were administered in two different doses: 7 mg and 15 mg per well. The compositions of the present invention (matrisome, NVDM2) showed a clear dose-dependent inhibitory impact on the migration ability of the OS cells, which was much stronger than in the presence of the gelatin beads alone (15 mg). Data represent the mean±s.d. values of % covered area at each time point normalized to the respective area at time point 0 h. Statistical significances between treated vs untreated samples at the respective time point were tested by ANOVA-I. **: p<0.01 and ns: not significant. Blue dots: values of individual batches (biological replicates). Each chart represents the average value of the data from all batches tested. DS: lyophilized non irradiated powder of NVDM2; DP: lyophilized powder of NVDM2 after irradiation.

FIG. 9 shows representative pictures showing the migration ability of 143B osteosarcoma cells in the absence/presence of gelatin beads through a scratch bioassay according to example 3. The migration rate of 143B cells was not impacted in the presence of the beads alone (15 mg). FIG. 9A shows untreated 143B cells at 0 h and at FIG. 9B at 24 h. FIG. 9C shows 143B cells treated with 15 mg gelatin beads at 0 h and at FIG. 9D at 24 h.

FIG. 10 shows representative pictures showing the migration ability of 143B osteosarcoma cells in the presence of the compositions of the present invention at two different doses through a scratch bioassay according to example 3. The migration of 143B cells was significantly inhibited by NVDM2 in a dose-dependent manner. FIG. 10A shows untreated 143B cells at 0 h and at FIG. 10B at 24 h treated with 7 mg of the composition of the present invention (NVDM2). FIG. 10C shows 143B cells treated with 15 mg of the composition of the present invention (NVDM2) at 0 h and at FIG. 10D at 24 h.

FIG. 11 shows the induction of early apoptosis in two osteosarcoma cell lines: 143B (FIG. 2A), U2OS (FIG. 2B), in comparison to human bone marrow MSCs (BM-MSCs) (FIG. 2C), in the presence of the compositions of the present invention (matrisome, NVDM2, Example 2) using Annexin V apoptosis Assay. Doxorubicin (1 uM and 5 uM) was used as a positive control treatment and gelatin beads alone were used for comparison with NVDM2 treatment. All treatments were carried out in 96-well plates in transwells. The compositions of the invention (NVDM2) were administered in two different doses: 7 mg and 15 mg per well. The viability was measured using Annexin V Apoptosis Assay at four different time points at 3, 6, 24 and 36 hours after administration. The compositions of the present invention (matrisome, NVDM2) showed a dose-dependent induction of apoptosis in the tested tumor cells which was significantly stronger than the one induced by gelatin beads alone (15 mg) (7-fold and 26-fold significantly higher levels of max apoptosis induction was observed after 6 h and 3 h of 15 mg of NVDM2 treatment in 143B and U2OS cells respectively, compared to treatment with 15 mg of beads alone. In contrast, only 2-fold (not significantly) higher max apoptosis induction was observed in hBM-MSCs upon 6 h of matrisome treatment compared to gelatin beads alone (15 mg).

Data represent the mean±s.d. values of fold induction normalized to the respective untreated sample. Statistical significances between treated samples by NVDM2 (15 mg) vs treated with beads alone (15 mg) at the respective time point were tested by Multiplied Unpaired T-test. *: p≤0.05, **: p<0.01 **: p<0.001 and ns: not significant. Blue dots: values of individual batches (biological replicates). Each chart represents the average value of the data from all batches tested.

FIG. 12 shows the Cell viability in DS (lyophilized powder) of matrisome in comparison to different quantities of the respective intermediate 3D cell product (CP), using the Cell Titer-Glo® luminescent cell viability assay. a) Correlation between the expected cellular content in the tested DS sample and in the different biopsies of CP (positive control samples). Based on the scheme shown in the graph, the cellular content in the tested biopsies of CP (29.1 mg-299.8 mg) corresponds to 0.5-6% of the expected total number of cells contained in the tested amount of DS, since 1990 mg of DS have been derived from the lyophilization of a CP biopsy weighing 4975 mg. (b) Luminescence values measured in 1990 mg of DS in comparison to the different biopsies of CP (Mean RLU for 1990 mg-DS:-328.8 whereas Mean RLU for 29.1 mg-CP DS: 1467.5). RLU: Relative Luminescence Units.

FIG. 13 shows in Volcano plot the proteomic data obtained for the compositions of the present invention (matrisome, NVDM2, Example 2) using Mass Spectrometry. (A) The red points indicate the different proteins that display both large magnitude fold-changes (x axis) during the manufacturing process (ASC before differentiation vs NVDM2 powder after irradiation, example 2) and high statistical significance (−log 10 of p values, y axis). Dashed horizontal line shows the p values cutoff, and the two vertical dashed lines indicate down/up regulated proteins. Transparent points in the significant region mean these proteins do not satisfy the selection conditions. (B) In this figure, the blue points indicate the 100 proteins that have been identified as the most abundant in NVDM2, among which EMILIN1, DCN, HTRA1, TNFSF10 and SFRP1 are ECM (extracellular matrix) proteins are known for their tumor suppressive properties and seem to be highly enriched during the manufacturing process of NVDM2. The following increases were observed:
EMILIN1 (471.5-fold enriched in NVDM2 vs ASCs)
DCN (164-fold enriched in NVDM2 vs ASCs)
HTRA1 (226-fold enriched in NVDM2 vs ASCs)
TNFSF10 (8-fold enriched in NVDM2 vs ASCs)
SFRP1 (52-fold enriched in NVDM2 vs ASCs)

### EXAMPLES

### Example 1: Manufacturing of the Composition of the Invention

NVDM2 is an allogenic/off-the shelf product, currently developed by Novadip Biosciences: and is derived from the autologous product NVD002 process. NVD-002 is produced from human Adipose-derived Stem Cells (hASCs) differentiated into osteogenic cells combined with gelatine particles derived from porcine skin (Cultispher) to produce a «scaffold-free» 3D grafts.

### 1.1 Upstream Process:

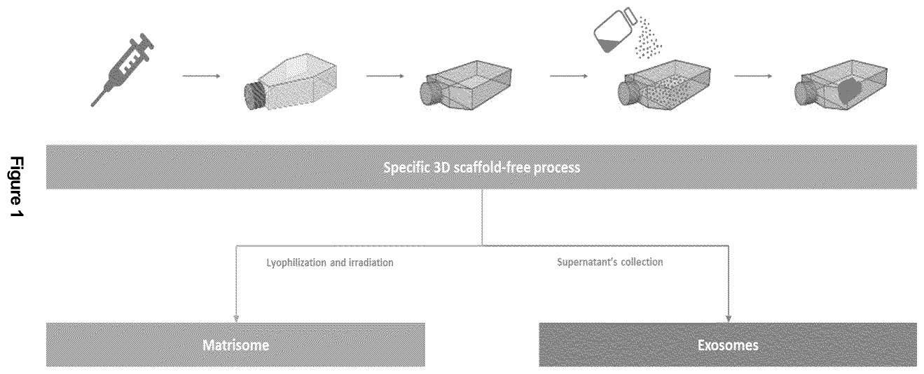

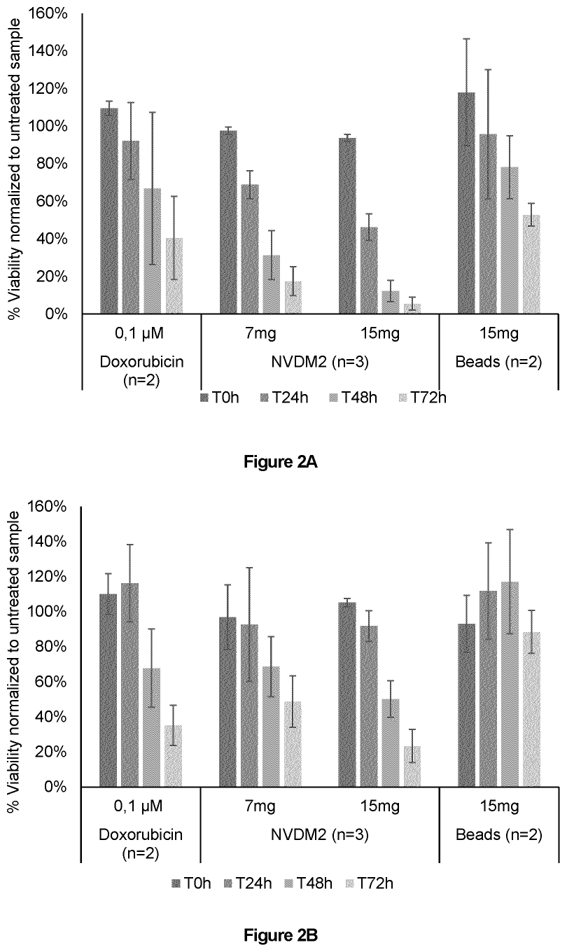

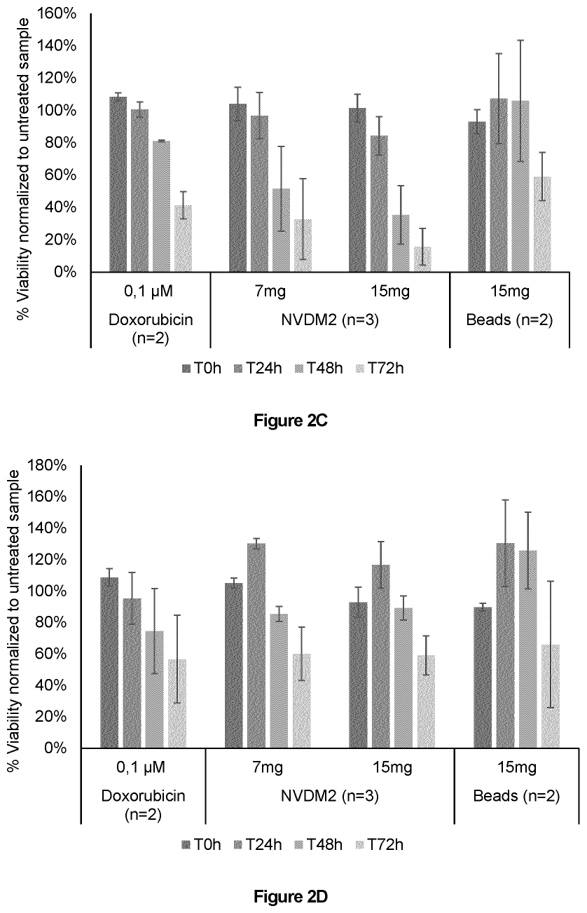

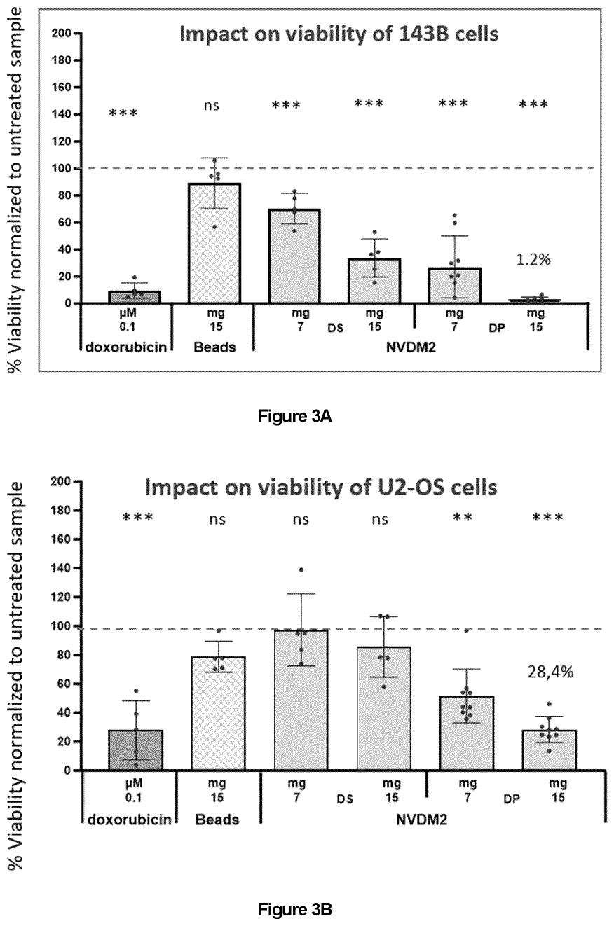

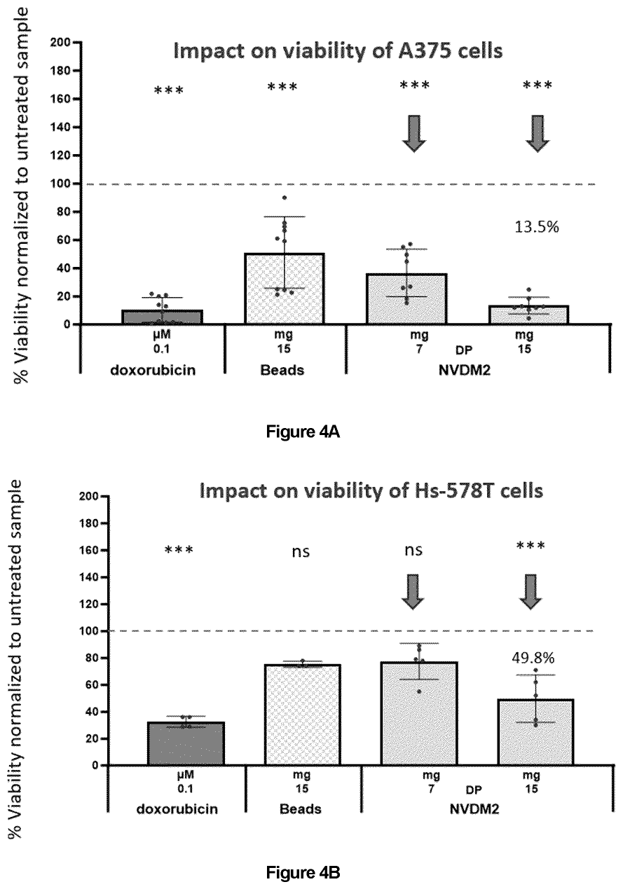

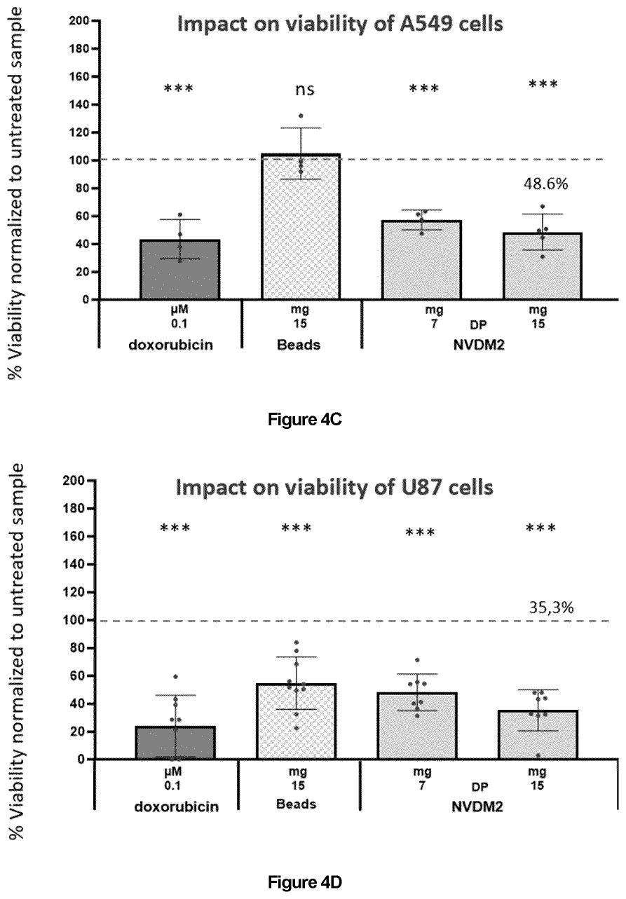

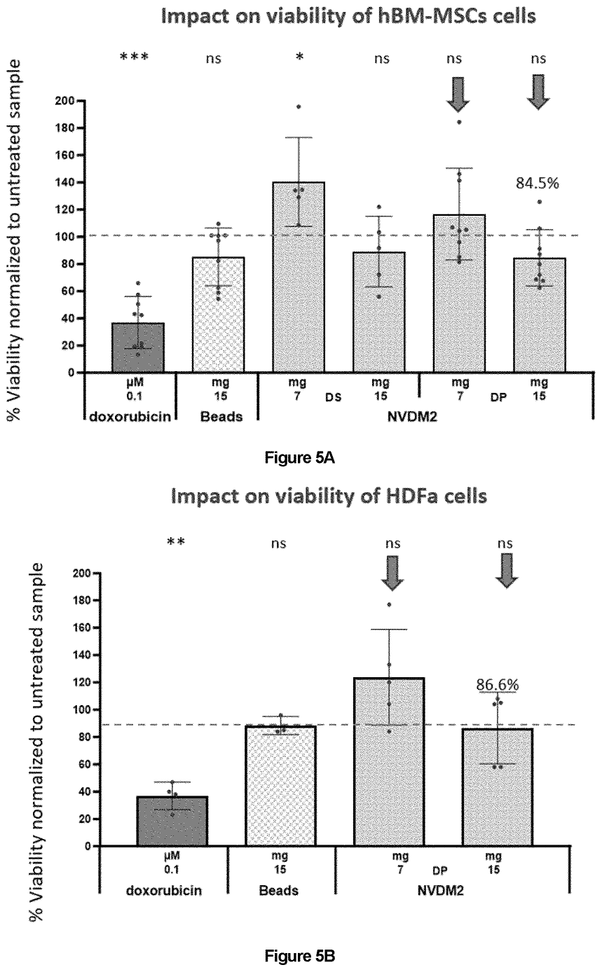

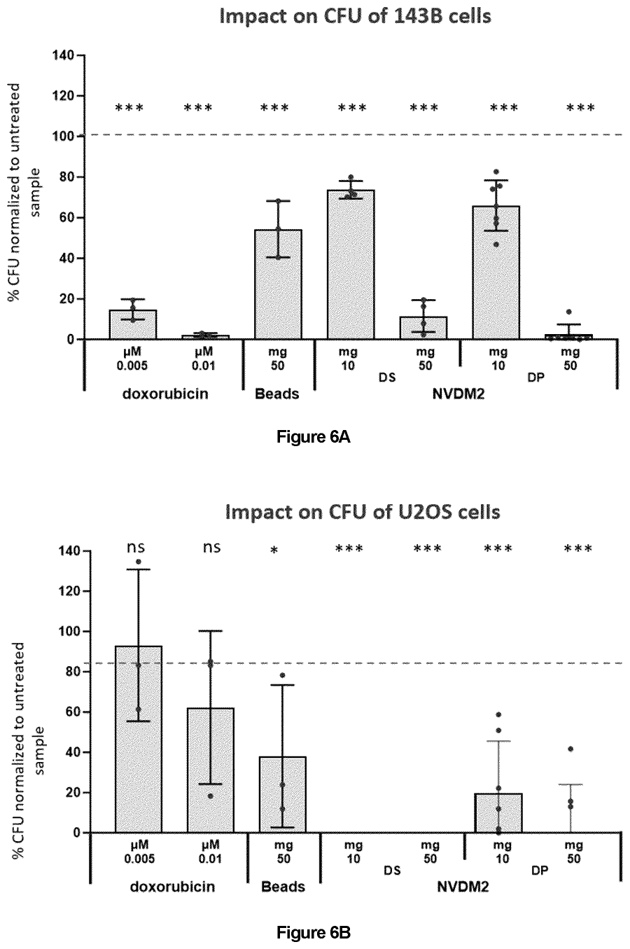

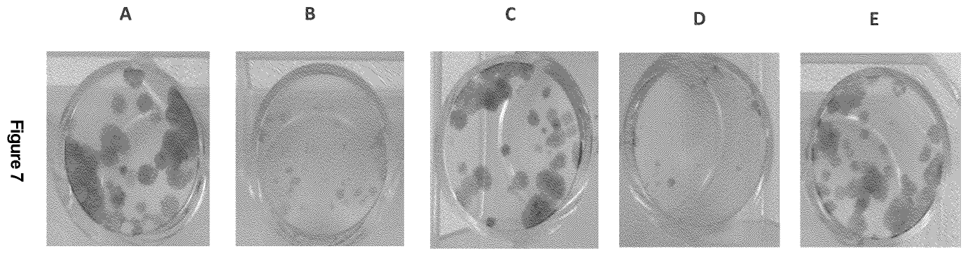

The manufacturing process of the active substance starts after the tissue procurement as shown in FIG. 1 comprising:
Stem cell collection;
Stem cell proliferation;
Osteogenic differentiation;
Particles sprinkling; and
3D-structure formation

The upstream process consists of the following 3 phases:

### 1.1.1 Upstream Process—Phase 1:

Isolation of the stromal vascular fraction (SVF) cells from the adipose tissue and subsequent expansion of human adipose-derived stem cells (hASCs) up to passage p3/p4 in proliferative medium (MP). The cells are expanded to cell stocks. The cell stock is cryopreserved.
   1.1.2 Upstream Process—Phase 2: Manufacturing of Three-Dimensional Cell Product which Includes the Following Steps:
Thawing of ASCs: The first step of NVDM2 cell products manufacturing process is the cell thawing at P4 from the cell stock. Cells are thawed and inoculated into 150-cm² flasks in proliferation medium and rinsed the day after.
Proliferation phase: After that, the cells proliferate until they reach a confluency ≥70% and ≤100%, before performing the passage P4/P5.
Passage P4/P5 and osteogenic induction phase: At the passage P4/P5, the cells collected from all the flasks are pooled and then seeded in T150 cm² flasks with re-closable lid (“TPP” flask) in osteogenic differentiation medium (MD) at a cell seeding density between 0,5×104 cell/cm² and 0,8×104 cell/cm².
When cells reach a confluency and at least one osteoid nodule (un-mineralized, organic portion of the bone matrix that forms prior to the maturation of bone tissue) is observed in each flask, the addition of gelatin beads can be launched.
Addition of Cultispher particles: After being exposed to the osteogenic differentiation medium (MD), the culture vessels containing the confluent monolayer of adherent osteogenic cells are sprinkled with gelatin beads.

### 1.1.3 Three-Dimensional Induction Phase:

Few days after the addition of the gelatin beads, the differentiated cells and the particles dispersed become progressively entombed in mineralizing the neo-synthesized extracellular matrix. At this point, the differentiated cells and gelatin beads particles start forming a large 3-dimensional patch (or few smaller patches) of partially mineralized brownish-yellow moldable putty detaching from each culture vessels.
The formation of the final three-dimensional cell product formation is obtained at the end of a maturation period. This cell product (CP) is frozen before downstream processing.

### 1.2 Downstream Process: Manufacturing of the Final Product Containing Non-Viable Cells.

During this phase of the manufacturing, lyophilization of all 3D-structures, grinding and mixing is occurred to produce the batch of Drug Substance (DS). The product is terminally sterilized by gamma-irradiation in order to obtain the Drug Product (DP).

### Example 2: Potency Bioassays to Assess the Anti-Tumor Activity of the Product on Osteosarcoma Cell Lines

The anti-tumor activity of matrisome was tested on three stable osteosarcoma cells lines: 143B, U2OS and SaOS-2. Human bone marrow mesenchymal cells (hBM-MSCs) were also used in the study to investigate whether the potential anti-proliferative effect of matrisome is targeted only to OS cells.

### Cell Culture

143B cells were maintained in MEM medium containing 10% foetal bovine serum (FBS), with penicillin, streptomycin and amphotericin B. U2OS, SaOS-2 and hBM-MSC were maintained in DMEM medium containing 10% fetal bovine serum (FBS), with penicillin, streptomycin and amphotericin B.

### Viability Bioassays

Cell viability was measured using the following two assays:

### Bioluminescence Cell Viability Assay

We used the Cell Viability Assay Kit (RealTime-Glo™ MT Cell Viability Assay) provided by Promega which employs the reducing potential of viable cells.

Cells were seeded in a 96-wells plate and allowed to attach for 24 h.

After O/N incubation, the medium was removed, and the cells were incubated for 72 h in the presence of the different doses of the test item. Doxorubicin treatment was also applied to each cell line as a reference compound to demonstrate that the system is able to detect inhibition of viability.

The treatments were carried out in transwells, in triplicates. Bioluminescence was measured at 0 h, 24 h, 48 h an 72 h after the initiation of the treatment to observe the time-dependent cytotoxicity.

### CCK-8 Assay

For this assay the Cell Counting Kit-8 Kit was used (Sigma-Aldrich). This viability assay is based on the reduction of tetrazolium salt (WST-8) by dehydrogenases within cells which gives an orange product soluble in the cell culture medium. This coloured product (formazan) is measured using a spectrophotometer. The formazan is directly proportional with the number of living cells presents in the cell culture.

Cells were seeded in 96-wells plates and allowed to attach for 24 h.

After O/N incubation, the medium was removed, and the cells were incubated for 72 h in the presence of the different doses of the test item. Doxorubicin treatment was also applied to each cell line as a reference compound to demonstrate that the system is able to detect inhibition of viability. The treatments were carried out in transwells, in triplicates. After 72 h of incubation, the 96-wells plate was removed from the incubator, washed with 2×PBS and 200 μl of medium containing 20 μl of CCK8 reagent was added in each well. The plate was incubated at 37° C. for 2 hour and then absorbance at 450 nm was measured.

### Example 3: Wound Healing Bioassays (Scratch Test)

143B, U2OS, SAOS-2 and hBM-MSC cells were seeded in 96-wells plate and grown to confluence.

The monolayer of the cells was scratched with the Incucyte® WoundMaker (96-pin mechanical device) and after two washes with PBS, the wounded cellular monolayer was exposed to 300 μl/well of medium with/without the tested doses of the test item. Doxorubicin treatment was also applied to each cell line as a reference compound to demonstrate that the system is able to detect inhibition of migration. All treatments were carried out in duplicates. Pictures of central wound edges per condition were taken at time point 0 h, time point 2 h, time point 4 h, time point 6 h, time point 24 h after the initiation of the treatment using Incucyte® at 10× magnification.

### Example 4: Cell Colony Formation Bioassays

143B, U2OS, SAOS-2 cells were cultivated in 24-wells plates and after 24 hours of culture, matrisome treatments were applied on the cells for 3-6 days in transwells, depending on the colony forming rate of each tested cell line. All treatments were carried out in duplicates. Doxorubicin treatment was also applied to each cell line as a reference compound to demonstrate that the system is able to detect inhibition of colony formation.

At the end of the treatments, medium was removed from all wells and proliferative colonies were incubated with fixative solution (10% acetic acid/10% methanol in DI water) for 15 minutes. Fixative solution was removed, and each well was incubated 500 μL of crystal violet 1% for 1 hour.

At the end of the incubation time, the staining solution was removed, and the plate was gently rinsed 3 times in a bath with DI water. When the plate was fully dried, pictures of each well were taken with a microscope. 500 μL of methanol were added to each well and incubated for 1 h for the sufficient dissolution of the staining dye and the optical density was measured at 570 nm.

### Example 5: Cell Viability in Matrisome

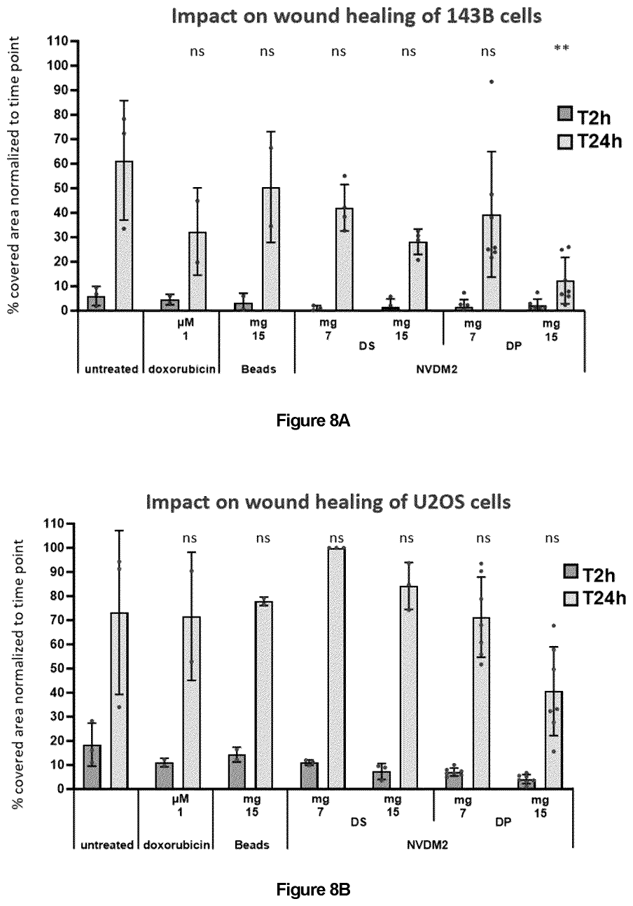

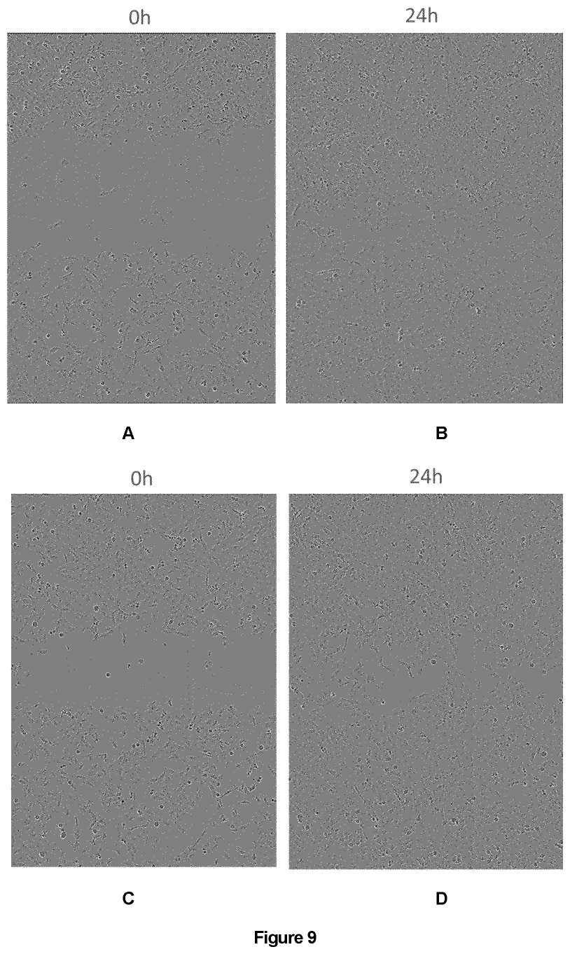

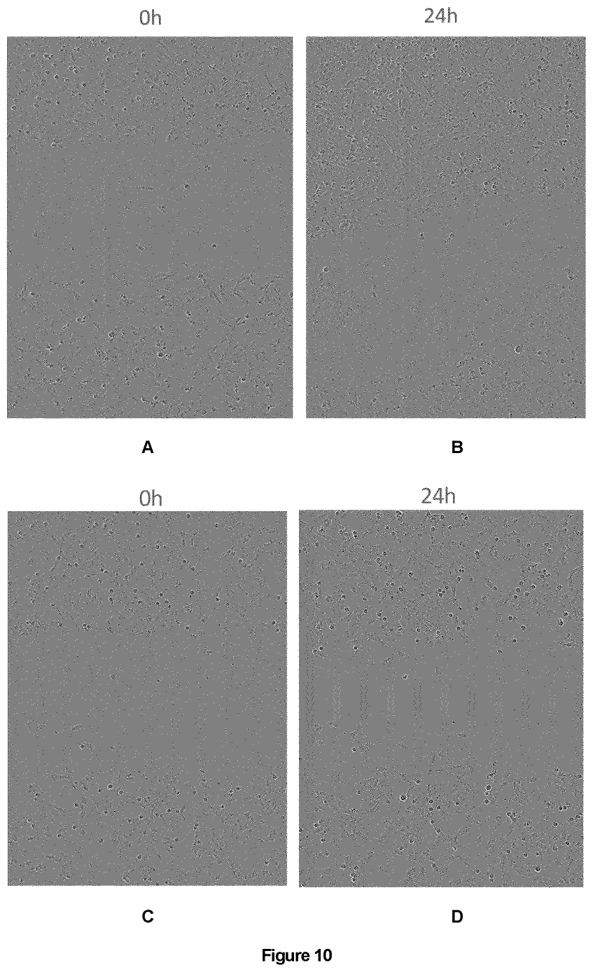

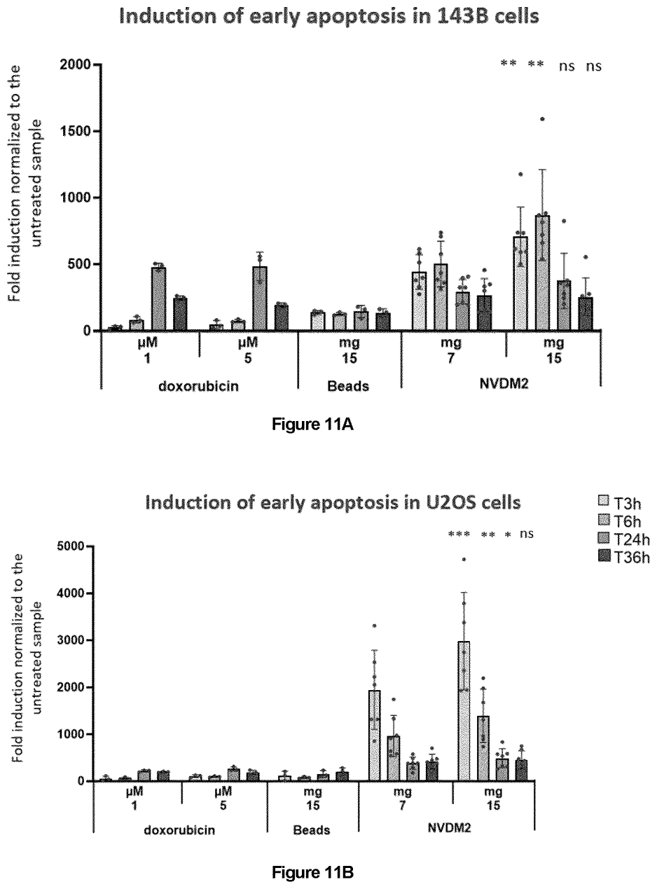

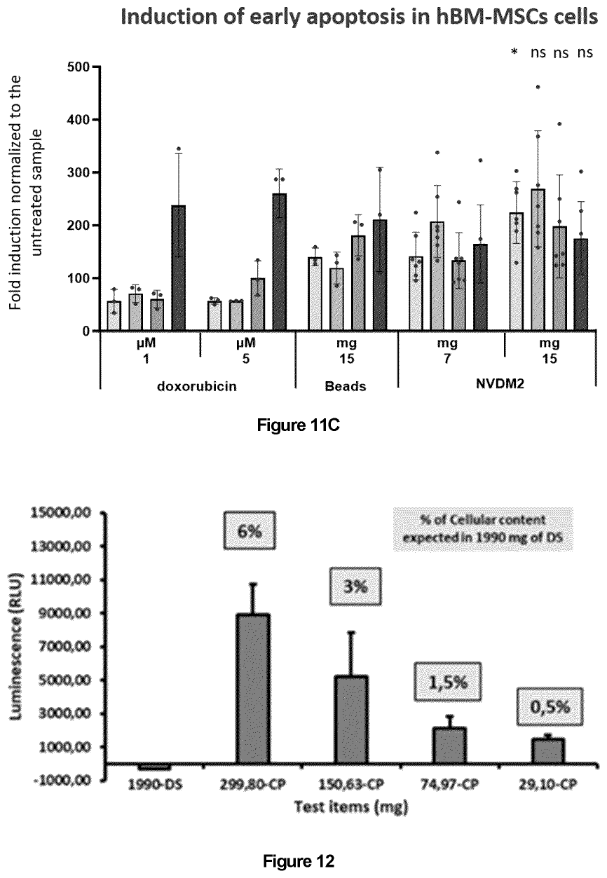

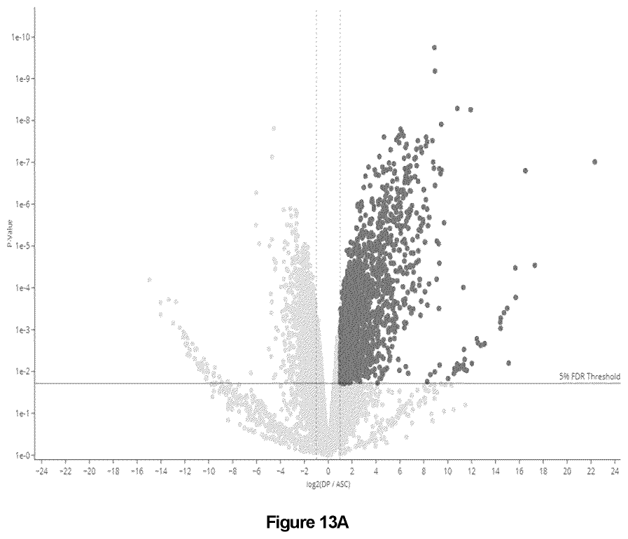

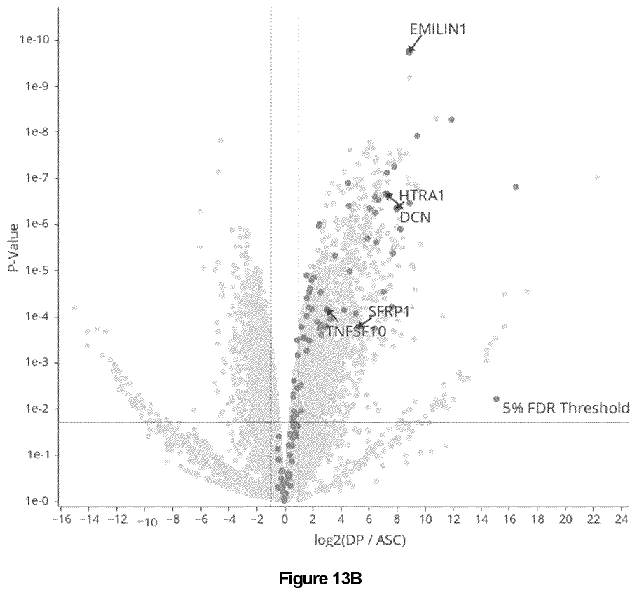

Following freeze-drying and terminal sterilization by -irradiation, matrisome is expected to be free of viable cells. A culture-based quantification of the ATP present inside the cells reflecting the cellular metabolic activity has been set-up. Data were reported to the weights of the respective autologous cell product samples knowing to contain metabolic active cells (FIG. 8).

In contrast to all the tested CP biopsies, no detectable values of metabolic activity have been obtained for all tested DS samples. Moreover, measurement of cellular metabolic activity in DS, based on the quantification of cellular ATP content, revealed values of cell viability lower than the limit of detection of the method which corresponds to the value detected in a biopsy of CP containing a number of cells equal to 0.5-0.6% of the expected cellular content in the tested DS.

This result shows the absence of viable cells in the lyophilized form of matrisome. The results were confirmed by additional tests performed to evaluate the presence of proliferative cells in the product or a potential release or consumption of key cellular metabolites by the product (such as glucose consumption or lactate production) (data not shown).

In conclusion, no viable or metabolically active cells have been detected in matrisome.

1. A composition comprising:
devitalized differentiated cells;
3-dimensional neo-synthesized extracellular matrix as a vehicle of bioactive compounds selected from the group consisting of proteins, mRNAs, miRNAs, lipids, extracellular vesicles proteins, mRNAs, miRNAs, lipids, extracellular vesicles; and
a particulate material;
for use in the treatment or prevention of cancer;
characterized in that:
the differentiated cells secreted the 3-dimensional neo-synthesized extracellular matrix prior to devitalization;
the cells and the particulate material are embedded in the 3-dimensional neo-synthesized extracellular matrix;
the composition is substantially free of extracellular vesicles; and
the particulate material is gelatin, and even more preferably gelatin beads.

2. The composition for use according to claim 1, wherein the composition comprises one or more of EMILIN1, DCN, HTRA1, TNFSF10, SFRP1.

3. The composition for use according to claim 1, wherein the composition comprises the bioactive compounds in an amount of twice the amount, preferably five times the amount as compared to 2D culture of undifferentiated cells, preferably to 2D culture of undifferentiated ASCs.

4. The composition for use according to claim 1, wherein the composition is used for:
inhibiting the viability of cancer cells,
inhibiting the proliferation of cancer cells;
inhibiting the migration of cancer cells;
inhibiting the cell colony formation of cancer cells;
or any combination thereof.

5. The composition for use according to claim 1, wherein the composition is:
desiccated, preferably by lyophilization size reduced, preferably by grinding to volumetric particle size distribution with a range of 100 to 5000 micrometers as measured by laser diffraction granulometry; and/or
sterilized, preferably by gamma-irradiation.

6. The composition for use according to claim 1, wherein the composition is free of external scaffolds.

7. The composition for use according to claim 1, wherein the differentiated cells are selected from the group comprising or consisting of osteoblasts, osteocytes, chondroblasts, chondrocytes, keratinocytes, myofibroblasts, epithelial cells, endothelial cells, adipocytes, neural cells, and precursors thereof, and preferably are soft tissue cells, chondroblasts or osteoblasts.

8. The composition for use according to claim 1, wherein the composition comprises the neo-synthesized extracellular matrix in a content of 0.001 w % to 10 w %, preferably from 0.01 w % to 7.5 w %, even more preferably from 0.1 w % to 5 w % as compared to the total weight of the composition.

9. The composition for use according to claim 1, wherein the devitalized differentiated cells are derived from stem cells selected from the group consisting of:
pluripotent stem cells (PSCs) such as embryonic stem cells (ESCs) or induced pluripotent stem cells (iPSCs);
adult stem cells such as hematopoietic stem cells (HSCs), skin stem cells (SSCs), neural stem cells (NSCs); and
mesenchymal stem cells (MSCs), preferably derivable from adipose tissue, peripheral blood or placenta,
and preferably are mesenchymal stem cells.

10. The composition for use according to claim 1, wherein the stem cells are derived from mesenchymal stromal cells, preferably obtainable from bone marrow, adipose tissue, placenta, or blood.

11. The composition for use according to claim 1, wherein the particulate material is selected from the group consisting of
an organic material, including demineralized bone matrix (DBM), gelatin, agar/agarose, alginates chitosan, chondroitin sulfate, collagen, elastin or elastin-like peptides (ELP), fibrinogen, fibrin, fibronectin, proteoglycans, heparan sulfate proteoglycans, hyaluronic acid, polysaccharides, laminins and cellulose derivatives;
a calcium compound;
a polymer, including polyanhydrides, polylactic acid (PLA), poly(lactic-co-glycolic acid) (PLGA), polyethylene oxide/polyethylene glycol (PEO/PEG), poly(vinyl alcohol) (PVA), fumarate-based polymers such as, for example polypropylene fumarate) (PPF) or polypropylene fumarate-co-ethylene glycol) (P(PF-co-EG)), oligopolypthylene glycol) fumarate) (OPF), poly(aldehyde guluronate) (PAG), polyp-vinyl pyrrolidone) (PNVP), or combinations thereof;
a gel, including a self-assembling oligopeptide gel, a microgel, a nanogel, a particulate gel, a hydrogel, a thixotropic gel, a xerogel, a responsive gel, or combinations thereof; and
a creamer; and
any combinations thereof.

12. The composition for use according to claim 1, wherein the composition further comprises a pharmaceutically acceptable carrier.

13. The composition for use according to claim 1, wherein the cancer is a solid cancer selected from the group consisting of s selected from the group comprising, or consisting of, a bone cancer, a brain cancer, a skin cancer, a breast cancer, a cancer of the central nervous system, a cancer of the cervix, a cancer of the upper aero digestive tract, a colorectal cancer, an endometrial cancer, a germ cell cancer, a bladder cancer, a kidney cancer, a laryngeal cancer, a liver cancer, a lung cancer, a neuroblastoma, an esophageal cancer, an ovarian cancer, a pancreatic cancer, a pleural cancer, a prostate cancer, a retinoblastoma, a small intestine cancer, a soft tissue sarcoma, a stomach cancer, a testicular cancer and a thyroid cancer, and preferably is bone cancer or any metastases thereof or skin cancer.

14. The composition for use according to claim 1, wherein the composition is administered
as a local implant;
topically, preferably as a transdermal patch; or
by injection, preferably in the form of an injectable suspension.

15. Implant, transdermal patch or injectable suspension comprising the composition according to claim 1 for use in the treatment or prevention of cancer, in particular for use in the inhibition of the viability, proliferation and migration of cancer cells.
